(19) United States
(12) Patent Application Publication
(10) Pub. No.: US 2007/0207463 A1
(43) Pub. Date: Sep. 6, 2007

(54) Method

(76) Inventors: Ziduo Liu, Hubei (CN); Yuzhi Hong, Hubel (CN); Lianhul Zhang, Singapore (SG)

Correspondence Address: HUNTON & WILLIAMS/NEW YORK;INTELLECTUAL PROPERTY DEPT., 1900 K STREET, N.W., SUITE 1200, WASHINGTON, DC, 20006-1109, US

(21) Appl. No.: 10/595,902

(22) PCT Filed: Nov. 20, 2003
(86) PCT No.: PCT/SG03/00274

§ 371 Date: Jan. 10, 2007

(51) Int. Cl. C12Q 1/68; C12P 19/34

(52) U.S. Cl. 435/6; 435/91.2

(57) ABSTRACT

A method for enriching the GC base pair content of a DNA molecule the method comprising the steps of (a) providing a DNA template molecule in which at least some of the A residues are base paired with U residues and (b) replicating the DNA template molecule provided in step (a) under conditions in the replication reaction medium in which at least some of the U residues base pair with a G residue. Typically, the DNA template molecule in (a) is produced by replicating a first template DNA molecule in the presence of dUTP so that at least some of the T residues of the first template are replaced by U residues to form a second template molecule. Thus, a preferred method comprises the steps of (1) providing a first template DNA molecule, (2) replicating the first template DNA molecule in the presence of dUTP so that at least some of the T residues of the first template are replaced by U residues to form a second template molecule and (3) replicating the DNA template molecule produced in step (2) under conditions in the replication reaction medium in which at least some of the U residues base pair with a G residue. The invention also includes a method for making a mutant polypeptide with altered properties compared to the polypeptide encoded by a DNA molecule, the method comprising (a) providing a DNA molecule encoding a polypeptide of interest, (b) enriching the GC base pair content of the DNA molecule according to the method set out above, (c) expressing the polypeptide encoded by the DNA molecule whose GC base pair content has been enriched and (d) selecting a polypeptide with altered properties. The invention also includes A mutant AlbD polypeptide wherein Ser40 has been replaced by another amino acid residue.

### METHOD

The present invention relates to a method for enriching the GC content of a DNA molecule and it further relates to the production of DNA molecules that encode a polypeptide with altered properties compared to a naturally encoded polypeptide.

The listing or discussion of a prior-published document in this specification should not necessarily be taken as an acknowledgement that the document is part of the state of the art or is common general knowledge. All documents listed are hereby incorporated herein by reference.

The genes encoding the traits of biotechnological and industry importance, such as enzymes and antibodies, are frequently modified for transgenic and heterogeneous expression. A first common objective of gene modification is to change the nucleotide composition of the gene based on the codon usage pattern of target host (Perlak et al., 1991; Narum et al., 2001; Valencik and McDonald, 2001, Shimshek et al., 2002; Yadava and Ockenhouse, 2003). This process usually does not change the amino acid composition of the gene product. Large scale genome sequencing revealed that there are remarkable divergences in nucleotide composition among different organisms (Ou et al., 2003; Tredj et al., 2002). Significantly, many microorganisms are low in GC content, while multicellular eukaryotes are generally GC-rich, especially at the 3^{rd} position of codon (Table1). Genome sequence analysis showed that warm-blooded animals and monocot plants have strong base compositional heterogeneity in genomes, consisting of relatively homogeneous regions, called isochores (>300 kb in size), which differ in GC content and gene concentration. It is believed that the heavy (GC-rich and gene rich) isochores were involved at later stages (Bemardi, 2000). Evidence suggests that GC-rich regions are more active in transcription in plants and animals because their superior bendability and B-Z transition ability favor open chromatin conformation, whereas AT-rich regions attract chromatin condensation that hinders transcription (Herbert and Rich, 1999; Vinogradov, 2003). This is consistent with the findings that heterogeneous AT-rich genes are commonly expressed poorly in higher organisms (Perlak et al., 1991; Narum et al., 2001; Shimshek et al., 2002).

TABLE 1
Comparison of GC content in the coding region of different
organisms*

| | Codon GC content (%) | | | |
|---|---|---|---|---|
| Organism | 1^{st} letter | 2^{nd} letter | 3^{rd} letter | Coding region |
| E. coli | 58.89 | 40.72 | 55.79 | 51.80 |
| Bacillus | 46.77 | 37.57 | 25.37 | 36.57 |
| thuringiensis | | | | |
| Candida | 43.96 | 37.53 | 28.74 | 36.74 |
| albicans | | | | |
| Plasmodium | 32.07 | 22.23 | 17.23 | 23.84 |
| falciparum | | | | |
| Saccharomyce | 44.53 | 36.55 | 37.85 | 39.64 |
| s. cerevisiae | | | | |
| Arabidopsis | 50.93 | 40.49 | 42.33 | 44.58 |
| thaliana | | | | |
| Maize | 56.66 | 42.79 | 60.50 | 53.32 |
| Rice | 58.46 | 46.43 | 61.61 | 55.50 |
| Sugarcane | 59.00 | 40.28 | 65.57 | 55.62 |
| Mouse | 55.62 | 42.22 | 59.30 | 52.38 |

*The data were based on the Codon Usage Tabulated from the Genbank website (www.kazusa.or.jp/codon/).

A second objective of gene modification is to improve enzyme/protein properties through directed protein evolution approaches (Stemmer, 1994; Crameri et al., 1998; Leung et al., 1989; Spee et al., 1993; Zaccolo et al., 1996) which are based on random change of the nucleotide and peptide composition. Directed evolution technologies have been revolutionizing the field of protein engineering, not only by producing the modified enzyme with improved activities (Glieder et al., 2002; Xia et al., 2002; Zaccolo et al., 1999; Zhang et al., 1997), thermostablity (Chemy et al., 1999; Giver et al., 1998; Miyazaki et al., 2000; Flores et al., 2002; Wintrode et al., 2003), substrate specificity (Leong et al., 2003; Yano et al., 1998; Rothman et al., 2003), and protein solubility (Yang et al., 2003), but also contributing significantly to the understanding of structural and functional relationships of proteins. Some of this progress has been attributed to the development of two types of key methodologies associated with PCR. The first is DNA shuffling (Stemmer et al., 1994; Crameri et al., 1998) and its derivative methods such as ITCHY (Ostermeier et al., 1999), STEP (Zhao et al., 1998), SliPE (Buchholz et al., 2001), degenerate homoduplex gene family recombination (Coco et al., 2002), and synthetic shuffling (Ness et al., 2002). The other is the random mutagenesis by changing the fidelity of DNA polymerase using Mn⁺⁺ (Leung et al., 1989), nucleoside analogues (Spee et al., 1993), or nucleoside derivatives (Zaccolo et al., 1996). These random mutagenesis methods are unable to promote AT to GC transition, except one error-prone PCR approach using unbalanced dNTP and high concentrations of Mn⁺⁺ and Mg⁺⁺ to force AT to GC transition (Fromant et al., 1995). However, the presence of Mn⁺⁺ also caused frameshift errors due to nucleotide deletion and insertion (Fromant et al., 1995), which diminished the chance of obtaining functional variants. Its application was further constrained by the finding that the DNA fragments longer than ˜400 bp could not be amplified in the presence of high concentration of Mg⁺⁺ (Fromant et al., 1995).

The AlbD protein of Pantoea dispera SB1403 is a carboxyl esterase that digests albicidin, a phytotoxin produced by a xylem-invading pathogen, Xanthomonas albilineans (Zhang & Birch, 1997). The transgenic sugarcane plants expressing high level of AlbD did not develop chlorotic disease symptoms in inoculated leaves, whereas all untransformed control plants and the transgenic plants expressing low level of AlbD developed typical symptoms (Zhang et al., 1999). However, the overall expression level of AlbD in transgenic sugarcane was very low (Zhang et al., 1999). The poor expression of AlbD, especially at the stem apex that is the key route of systemic infection, might account for the less satisfactory performance of transgenic sugarcane against systemic infection of X albilineans in field trial (Zhang and Birch, unpublished data). A solution to improve AlbD performance is to modify albD, either by altering the nucleotide composition following the high GC content pattern of sugarcane (Table 1), or by enhancing the catalytic efficiency of the AlbD enzyme.

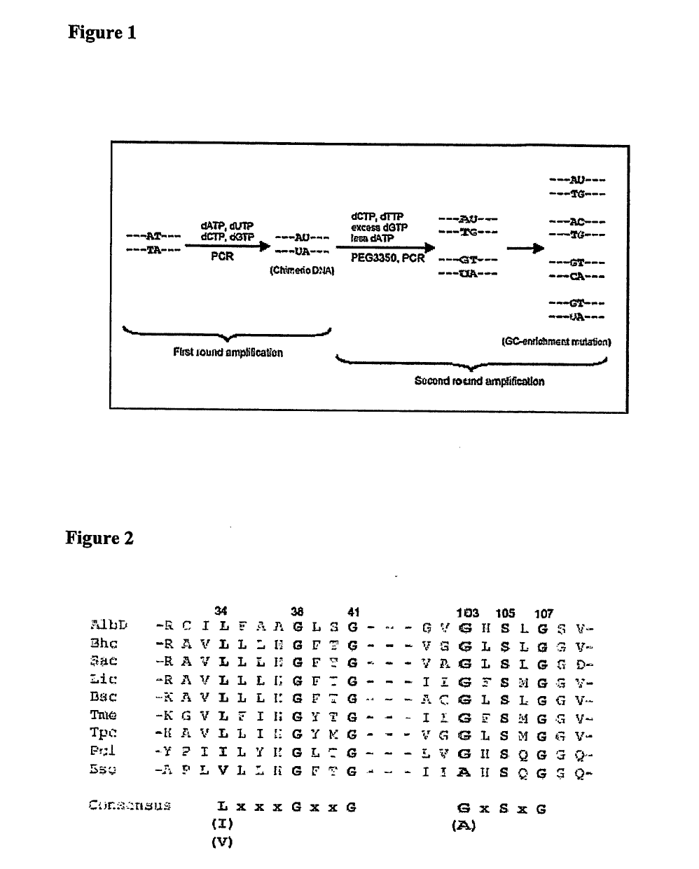

In the study described in Example 1, we have established a new approach of mutagenesis to combine GC-enrichment and directed protein evolution as one method. Instead of random substitution of nucleotides, we exploited the nature that dUMP can conveniently replace dTTP and pair with dGMP under certain conditions to promote AT to GC conversion (FIG. 1). We show that this GC-enrichment protocol is also effective in generating evolved enzymes with improved catalytic properties. The method could thus be used as an in vitro functional alternative to the natural evolution process which was believed to account for the emergence of GC-rich isochores in warm-blooded animals and monocot plants (Galtier et al., 2001; Vinogradov, 2003).

It will be appreciated that the first step of the approach can be used to enrich the GC base pair content of any suitable DNA molecule, but in a particular embodiment the enrichment leads to a change(s) in codon(s) of the DNA molecule so that different amino acids are encoded which means that the resultant DNA molecule may encode a polypeptide with altered, typically improved, properties. The method may also be used to select GC base pair-enriched molecules which retain the same coding sequence as the parent DNA molecule, but have improved codon usage for expression in eukaryotes, especially higher eukaryotes, as well as in the microorganisms with GC-rich genomes.

Thus, a first aspect of the invention provides a method for enriching the GC base pair content of a DNA molecule the method comprising the steps of (a) providing a DNA template molecule in which at least some of the A residues are base paired with U residues and (b) replicating the DNA template molecule provided in step (a) under conditions in the replication reaction medium in which at least some of the U residues base pair with a G residue.

It will be appreciated that by encouraging the U residues to base pair with an incoming G residue in the DNA strand that is being synthesised in the replication process, A residues are being replaced by G residues. Further replication of the DNA containing the G-U base pair will fix the mutation in at least some of the resulting DNA molecules such that the effect is to cause an AT to GC transition mutation in the parent DNA molecule.

Conveniently, the DNA template molecule in (a) is produced by replicating a first template DNA molecule in the presence of dUTP so that at least some, preferably all of the T residues of the first template are replaced by U residues to form a second template molecule. In other words, dUTP is present in the first replication reaction medium for producing the second DNA template from the first DNA template along with dATP, dCTP and dGTP, whereas dTTP is typically absent (but may be present alongside dUTP). Typical concentrations of the deoxynucleotides used in this reaction are 200 μM each of dATP, dGTP and dCTP and 500 μM dUTP, but any suitable concentrations may be used although typically there is an excess of dUTP.

Thus, in a particularly preferred embodiment of the invention the method comprises the steps of (1) providing a first template DNA molecule, (2) replicating the first template DNA molecule in the presence of dUTP so that at least some, preferably all of the T residues of the first template are replaced by U residues to form a second template molecule and (3) replicating the DNA template molecule produced in step (2) under conditions in the replication reaction medium in which at least some of the U residues base pair with a G residue.

The first template DNA molecule is one in which it is desirable to enrich the GC base pairs, and examples of such molecules are given below.

In step (3) above, conditions are produced in the replication reaction medium which favour the base pairing of the U residue in the template strand with an incoming G residue in the strand being synthesised rather than with an incoming A residue. Suitable conditions can be determined by analysing the products of the reaction by DNA sequencing to determine whether or not there has been an AT to GC transition mutation and, if so, how many such mutations. Conveniently, suitable conditions may be generated by using an agent in the second replication reaction medium which promotes the bringing together of the G and U bases. Typically the agent is one which increases the polarity (enhances the polar environment) of the replication reaction medium and/or which acts as a local, molecular dehydrating agent (which encourages the formation of G-U base pairs). A particular, suitable agent is polyethylene glycol (PEG), especially PEG 3500. PEGs from PEG300-PEG8000 can have similar effect when used in a suitable concentration. PEG300 means a PEG polymer with molecular weight of 300, PEG8000 has a molecular weight of 8000.

Another way of producing conditions which favour the formation of G-U base pairs in this step is the presence of a large excess of dGTP over dATP in the second replication reaction medium. Thus, typically, the second replication reaction medium contains 200 μM each of dCTP and dTTP, 600 μM dGTP and 12 μM dATP. Concentrations in the range of 20 μM-2000 μM may be used.

Typically, the second replication reaction medium has both an excess of dGTP over dATP, and contains an agent, such as PEG, which promotes the bringing together of the G and U bases to form a base pair.

Conveniently, each of the two replication reactions are polymerase chain reactions (PCR). Thus, in a particularly preferred embodiment of the invention, a template DNA molecule (eg a natural DNA template) is subjected to a first PCR reaction in the presence of dATP, dCTP, dGTP and dUTP in order to make a DNA molecule in which at least some, preferably all of the T residues on either strand are replaced by U residues. Typically, dUTP is present in a molar excess over dATP, dCTP or dGTP. Preferably, dATP, dCTP and dGTP are present at a concentration of 200 μM, whereas dUTP is present at a concentration of 500 μM. These concentrations may be altered by the skilled person without inventive effort, for example they may need to be varied if a DNA polymerase other than Taq is used in the PCR.

The PCR product containing dUMP (ie containing U residues) is then used as a template for a second PCR.

The second PCR is typically carried out in the presence of dATP, dCTP, dGTP and dTTP, but in this case there is a molar excess of dGTP over the other three deoxynucleotides, and a molar excess of dGTP, dCTP and dTTP over dATP. Preferably, dCTP and dTTP are present at 200 μM, dGTP is present at 600 μM and dATP is present at 12 μM. These concentrations may be altered by the skilled person without inventive effort, for example they may need to be varied if a DNA polymerase other than Taq is used in the PCR. MgCl₂ is typically present in the reaction medium, for example at a concentration of 3.5 mM.

The method of the invention does not appear to produce frameshifts, and it could be used on any length of DNA molecule which can be amplified by normal PCR reactions (for example a 15 kb gene has been amplified by PCR)

The method of the invention may be used to enrich the GC base pair content of any DNA molecule where this is desired. Although the method uses double stranded DNA, it will be appreciated that it may be applied to RNA molecules or single stranded DNA which have been converted into double stranded DNA molecules, for example by reverse transcription and cDNA synthesis. Typically the method is used to enrich the GC base pair content of DNA molecules which have a relatively low GC base pair content, or an undesirably low GC base pair content for the purpose to which the DNA molecule is to be put. In particular, as noted above for example in Table 1, certain genes from microorganisms have a significantly lower GC content than genes in higher eukaryotes, which may limit the ability of these genes to be expressed in higher eukaryotes.

It is particularly preferred if the GC base pair content of the DNA molecule to be so enriched is lower than 50% (for example lower than 45%, 40%, 35%, 30% or 25%, but the invention is also applicable to modify the genes with GC content higher than 50% as a minor change of GC content could result in significant improvement of enzyme properties, and such examples are given below.

Preferably, the DNA molecule whose GC base pair content is to be enriched is all or a part of a gene or a cDNA. More preferably, the gene or cDNA is one which has a GC base pair content of lower than 50% (though it may be higher, as noted above), preferably lower that 45% or 40% or 35% or 30% or 25%. It is particularly preferred if the gene or cDNA encoded a polypeptide of interest, particularly one where it is desired to produce mutants with altered properties. As is described below, a further embodiment of the invention is the production of mutant polypeptides.

It will be appreciated that a variety of DNA molecules will be produced by the method of the invention and unless the context indicates the contrary, the reference to a singular DNA molecule is a reference to more than one DNA molecule. In order to obtain a single DNA molecule, it is particularly convenient to clone the DNA molecule whose GC base pair content has been enriched. Methods of cloning are well known in the art and are described in detail in standard manuals such as Sambrook & Russell (2001) Molecular Cloning, a laboratory manual, Cold Spring Harbor Press, Cold Spring Harbor, N.Y., USA. When the DNA molecule whose GC base pair content has been enriched encodes a polypeptide, it is particularly useful to clone the DNA molecule into an expression vector. The expression vector suitably contains the necessary transcription and translation control elements to enable the encoded polypeptide to be expressed in a chosen host cell. Thus, the DNA molecule may be cloned into a mammalian expression vector or into a plant expression vector or into a prokaryotic vector.

Typical prokaryotic vector plasmids are: pUC18, pUC19, pBR322 and pBR329 available from Biorad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA).

A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems (La Jolla, Calif. 92037, USA). Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

Plant transformation vectors include Agrobacterium vectors, which deliver the DNA by infection. Other vectors include ballistic vectors and vectors suitable for DNA-mediated transformation. These methods are known to those skilled in the art. See, for example, the review by C. P. Lichtenstein and S. L. Fuller, “Vectors for the genetic engineering of plants”, Genetic Engineering, ed. P. W. J. Rigby, vol. 6, 104-171 (Academic Press Ltd. 1987).

The invention also includes DNA molecules enriched for GC base pair content prepared by the above methods, including those cloned into a vector, such as an expression vector.

In a further step of the method, the DNA molecule enriched for GC base pair content, whether cloned or not, is sequenced. This may be done using standard DNA sequencing technique, such as the Sanger dideoxy method.

It will be appreciated that sequencing the DNA molecule gives information concerning the coding sense of the molecule (if the molecule encodes a polypeptide). Thus, from the sequence, it is possible to determine whether the coding sense is retained (in which case the DNA molecule will encode the same polypeptide as the parent; this occurs generally by the AT to GC mutation occurring in the third base position of degenerate codons) or whether it has been altered (in which case the DNA molecule will encode a different polypeptide to the parent molecule, which may have different properties).

In some circumstances, it is desirable to retain the coding sense of the DNA molecule, for example when it is desired to express the same polypeptide as the parent molecule, but it is also desired for the GC base pair content to be increased so as to improve transcription or translation in certain host cells. In other circumstances it is desirable for the coding sense to be altered so that the DNA molecule encodes a polypeptide with altered properties.

A further embodiment of the invention provides a method for making a mutant polypeptide with altered properties compared to the polypeptide encoded by a given DNA molecule, the method comprising (a) enriching the GC base pair content of the DNA molecule according to the method of the first aspect of the invention, (b) expressing the polypeptide encoded by the DNA molecule whose GC base pair content has been enriched in step (a), and (c) selecting a polypeptide with altered properties.

It will be appreciated that in this embodiment, the DNA molecule whose GC base pair content is to be enriched is one which encodes a polypeptide and so typically is all or part of a gene or cDNA. The polypeptide is any polypeptide of interest whose properties it is desired to alter. Typically, the polypeptide is an enzyme or antibody or an antigen or an other type of therapeutic protein.

This method allows for the simultaneous enrichment of GC base pairs in a DNA molecule and production of DNA molecules with altered polypeptide coding potential.

The polypeptide may conveniently be selected for altered properties using methods well known in the art. Typically, the properties of the polypeptide which are altered are solubility, thermostability, catalytic activity (if the polypeptide is an enzyme), substrate specificity (if the polypeptide is an enzyme), protein stability, ligand affinity, and immunological properties and so on.

In the case of enzymes, it is particularly desirable to improve their catalytic properties and/or to change their substrate specificity. The improved enzyme can be selected either by monitoring the rate of substrate consumption or the speed of product formation. In some cases, the catalytic activity can be determined by the change of the cofactor properties, e.g., conversion of NAD⁺ to NADH or vice versa.

Other desired protein properties, such as thermostability, antibody affinity, and immunological properties can be selected based on well known techniques in the fields of biochemistry and immunology.

The invention also includes mutant polypeptides prepared according to the method of this embodiment of the invention. It will be appreciated that once a mutant has been selected and the sequence of the DNA molecule encoding it has been determined it will be possible to make the mutant by any standard protein engineering method, such as those including site-directed mutagenesis.

In a particular embodiment of the invention, the method was applied to the albD gene of Pantoea dispersa SB1043.

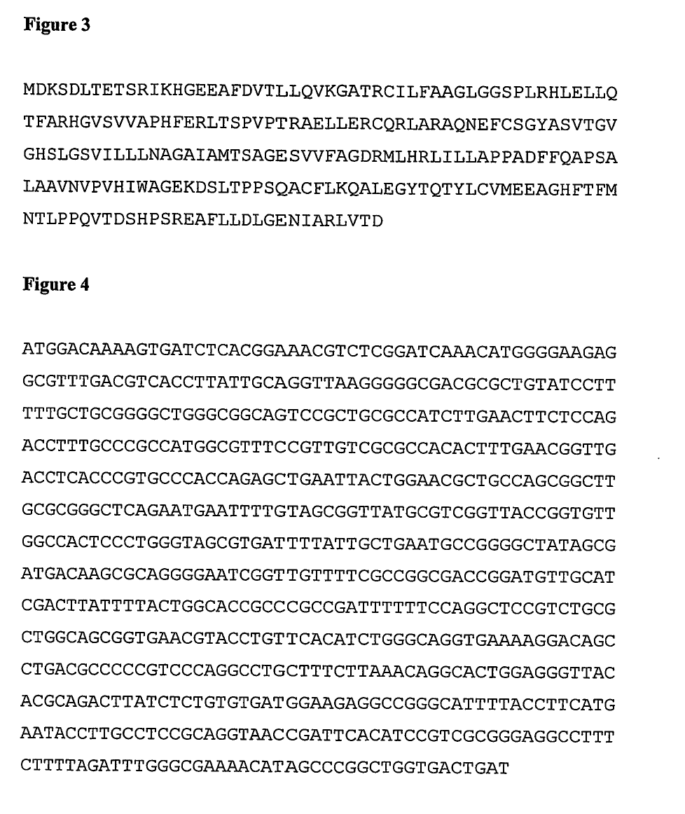

Two mutant enzymes were selected, one of which contains the mutation Ser40Gly (termed AlbD-M1), and the other contains the mutations Glu25Arg, Lys27Glu and Ser40Gly. The amino acid sequence of the AlbD-M1 mutant is given in FIG. 3, and the nucleotide sequence of the DNA molecule encoding it (albD-M1) is given in FIG. 4.

Thus, a second aspect of the invention provides a mutant AlbD polypeptide wherein Ser40 has been replaced by another amino acid residue. The amino acid which replaces Ser40 may be any amino acid. It is particularly preferred if Gly replaces Ser 40, since the albicidin detoxification activity of this mutant was increased 3-fold compared to wild-type. The mutant in which additionally Glu25 has been replaced by Arg, and Lys27 has been replaced by Glu is also preferred since the albicidin detoxification activity of this mutant was increased 1.7 fold compared to the wild-type. These mutant albicidin detoxifying enzymes are useful for detoxifying albicidin, for example when expressed transgenically in plants (see, for example, Zhang et al., 1999).

A third aspect of the invention therefore includes a polynucleotide encoding a mutant AlbD polypeptide wherein Ser40 has been replaced by another amino acid residue. In particular embodiments, the polynucleotide is contained within an expression vector, especially a plant expression vector. A further embodiment is a transgenic plant containing a polynucleotide which encodes a mutant AlbD polypeptide wherein Ser40 has been replaced by another amino acid residue. Vectors and transgenic plants can be made using methods well known in the art (see Zhang et al., 1999 for details).

Of course, in relation to the second and third aspects of the invention and embodiments thereof the amino acid sequence of the mutant AlbD polypeptide may differ from of a naturally occurring AlbD polypeptide at other positions than those indicated above. For example, the mutant AlbD polypeptide may differ at further positions from the sequence shown in FIG. 3. Variants (whether naturally-occurring or otherwise) may be made using the methods of protein engineering and site-directed mutagenesis well known in the art.

By “variants” of the polypeptide we include insertions, deletions and substitutions, either conservative or non-conservative. In particular we include variants of the polypeptide where such changes do not substantially alter the activity of the said polypeptide. In particular we include variants of the polypeptide where such changes do not substantially alter the activity, for example the activity as discussed above of the said polypeptide.

It will be appreciated that a variant that comprises substantially all of the sequence shown in FIG. 3 may be particularly useful. By “substantially all” is meant at least 80%, preferably 90%, still more preferably 95%, 98% or 100% (ie all) of the said sequence. By “substantially full-length” is meant comprising at least 80%, preferably 90%, still more preferably 95%, 98% or 100% (ie all) of the sequence of the full length polypeptide.

By “conservative substitutions” is intended combinations such as Gly, Ala; Val, Ile, Leu; Asp, Glu; Asn, Gln; Ser, Thr; Lys, Arg; and Phe, Tyr.

It is particularly preferred if the polypeptide variant has an amino acid sequence which has at least 65% identity with the amino acid sequence of naturally occurring AlbD (for example the sequence on which the mutants discussed above are based, for example as indicated in FIG. 3), more preferably at least 75%, still more preferably at least 90%, yet more preferably at least 95%, and most preferably at least 98% or 99% identity with the said amino acid sequence, most preferably with the amino acid sequence given in FIG. 3.

It is particularly preferred if the polypeptide variant has an amino acid sequence which has at least 90% identity with the amino acid sequence shown in FIG. 3, more preferably at least 92%, still more preferably at least 95%, yet more preferably at least 96%, and most preferably at least 98% or 99% identity with the said amino acid sequence.

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program (Thompson, J. D., Higgins, D. G. and Gibson, T. J. (1994), Clustal-W—improving the sensitivity of progressive multiple sequence alignment through sequence weighting, position specific gap penalties and weight matrix choice. Nuc. Acid Res. 22, 4673-4680).

The parameters used may be as follows:

Fast pairwise alignment parameters: K-tuple(word) size; 1, window size; 5, gap penalty; 3, number of top diagonals; 5. Scoring method: x percent.

Multiple alignment parameters: gap open penalty; 10, gap extension penalty; 0.05.

Scoring matrix: BLOSUM.

A fourth aspect of the invention provides a kit of parts for enriching the GC base pair content of a DNA molecule in a replication reaction medium comprising (a) dUTP and (b) an agent which is able to increase the polarity of the replication reaction medium and/or act as a local dehydrating agent.

Typically, the agent which is able to increase the polarity of the replication reaction medium and/or act as a local dehydrating agent is a polyethylene glycol, preferably PEG 3350.

Conveniently, the kit of parts further comprises other reagents for carrying out a DNA amplification reaction, such as dATP, dCTP, dGTP and dTTP, and a thermostable DNA polymerase such as Taq.

The invention will now be described in more detail by reference to the following non-limiting Figures and Example wherein:

FIG. 1 is a schematic diagram of GC-enticement mutagenesis. The first round of amplification is conducted in the presence of dUTP and the absence of dTTP. Chimeric PCR product is used as the template for the second round of amplification in the presence of excess amount of dGTP and minimal concentration of dATP.

FIG. 2 shows the LxxxGxxG and GxSxG regions of various esterases and lipases. Bhc, Bacillus halodurans carboxylesterase (Takami et al., 2000); Sac, Staphylococcus aureus N35 carboxylesterase (Kuroda et al., 2001); Lic, Listeria innocua carboxylesterase (Glaser et al., 2001); Bsc, Bacillus subtilis carboxylesterase (Kunst et al., 1997); Tme, Thermotoga maritimal esterase (Nelson et al., 1999); Tpc, Treponema pallidum carboxylesterase (Fraser et al., 1988); Pcl, Pseudomonas cepcia lipase (Derewenda & Sharp, 1993); Bse, Bacillus stearothermophilus esterase (Kugimiya et al., 1992).

FIG. 3 shows the amino acid sequence of AlbD-M1

FIG. 4 shows the DNA sequence of albD-M1

### EXAMPLE 1

### A GC-Enrichment Method for In Vitro Molecular Evolution of proteins and enzymes

Summary

The GC-rich genes of warm-blood animals and plants are in general more active in transcription than the AT-rich counterparts. Many microorganisms, however, are AT-rich in genome sequences. Here we describe a novel GC-enrichment mutagenesis protocol that employs dUTP to replace dTTP and promotes U:G mismatch, hence resulting in AT to GC conversion. We used this method to generate a mutant library of albD, which encodes a carboxyl esterase capable of degrading albicidin phytotoxin, a key virulence factor of the plant bacterial pathogen Xanthomonas albilinens. Among the evolved enzymes with enhanced activity, AlbD-M1 showed up to 43-fold increase in the catalytic efficiency (K_{cat}/Kₘ) over the wild type AlbD. Sequence analysis of the mutants led to identification of a “L(I,V)xxxGxxG” motif, which is widely conserved in lipases/esterases and associated with the catalytic oxyanion hole. These results indicate that combination of directed protein evolution with GC-content modification should be a useful approach, in particular, for modification of AT-rich genes for transgenic and heterologous expression.

Experimental Protocol

GC-enrichment mutagenesis. pQE60-GFP carrying a GFP gene was used as a template to generate a chimeric ‘DNA’ by PCR using forward primer 5′-GGTCCAGGAGG AAAAAGGC-3′ and reverse primer 5′-GTTCTGAGGTCATTACTGG-3′ (10 pmole primer each) in 50 μl reaction mixture containing 1×PCR buffer (Bio-Lab), 200 μM each of dATP, dGTP, and dCTP, 500 μM dUTP, 100 pmole template DNA, and 0.5 unit of Taq DNA polymerase (Bio-Lab). PCR was performed for 30 cycles consisting 3 minutes at 94° C. followed by 40 seconds at 94° C., 30 seconds at 50° C., and 50 seconds at 72° C. Then, the PCR product containing dUMP was used as a template for second round amplification using the same condition as described above, except that 3.5 mM MgCl₂ and 100 μM polyethylene glycol 3350 (PEG3350) were included in PCR buffer, dGTP concentration was increased to 600 μM, and dATP concentration was reduced to 12 μM unless otherwise indicated. The PCR product was digested by BamHI and HindIII and ligated to expression vector pQE60 (QIAGEN) and transformed into E. coli DH5α. The fluorescence phenotype of GFP provided a useful indication of the mutation frequency at the early optimization process.

Construction of AlbD mutation library and screening for transformants with enhanced albicidin resistance. pGST-albD carrying the albD gene was used as a template to generate a diverse mutation library by GC-enrichment mutagenesis method described above using forward primer 5′-CGCGTGGATCCGTTTGATGGACA-3′ and reverse primer 5′-GATGAATTCCCCTGGAAAAGCTTATCCC-3′. The PCR product was digested and inserted into pQE60, which were then transformed into the E. coli DH5α. The transformants were screened for enhanced albicidin resistance on LB plates containing a sub-lethal dose of albicidin against wild-type E. coli DH5α (pGST-albD). The colonies showing better growth on albicidin selection plates than E. coli DH5α (pGST-albD) were then selected for DNA sequence analysis and quantification of enzyme activity.

Purification of AlbD and variants. The coding sequence of albD was amplified by PCR using forward primer 5′-ATGGGAGGATCCTTTTGATGGACA-3′ and reverse primer 5′-CTCAGCGAATTCAGCTTATCCC-3′. The PCR product was digested by BamHI and EcoRI and fused in-frame to GST (glutathione S-transferase) gene in expression vector pGEX-2T (Pharmacia). E. coli DH5α containing the GST-AlbD fusion construct was grown in LB medium at 30° C. overnight. The cells were harvested by centrifuging at 5000 rpm for 10 minutes. The cells were resuspended in PBS buffer (pH7.4) and lysed by using a French Pressure Cell Press (Aim-Aminco) at 1100 psi. The lysate was centrifuged at 18000 rpm for 60 min at 4° C. The supernatant was loaded in the pre-equilibrated Gluthione Sepharose 4B affinity column and washed with PBS buffer (pH 7.4) to remove non-specifically bound proteins. AlbD was separated from GST and released from the affinity column by digestion with thrombin (Sigma-Aldrich) for 15 h at 4° C. The enzyme purity was determined using SDS-PAGE and stored at −80° C. in PBS buffer containing 50% glycerol. The AlbD variants were purified using the same method.

Assay of albicidin detoxification activity. Albicidin detoxification activity was determined by plate assay using E. coli DH5α as the indicator as described previously (Zhang et al., 1998). A 20 μl PBS buffer (pH7.4) containing AlbD (0.006˜0.025 μM/μl), and albicidin (15 ng/μl) was incubated at 28° C. for 5 min. The reaction was stopped by adding 10% SDS to a final concentration of 1%. The reaction mixture was added to the pre-punched wells (3 mm in diameter) on the bioassay plate and was incubated at 37° C. overnight. The remaining albicidin at the end of reaction was calculated by the formula: albicidin (ng m⁻¹)=4.576 e^{(0.135W)}, where W is the diameter of inhibition zone. AlbD activity is presented as the percentage of albicidin degraded by the enzyme.

Kinetics analysis of wild type AlbD and its variants. Enzyme kinetics of AlbD and variants were determined using p-nitrophenyl compounds, the commonly used esterase substrates. The reaction was conducted in 165 μl PBS buffer (pH 7.4) containing enzyme (0.3-0.6 μM), and 3 mM p-nitrophenayl compounds at room temperature for 5 minutes and O.D₄₀₅ was determined. For kinetics assay, we used the Kinetics program in the Athous spectrometer (Australia) to obtain slopes with different enzyme concentrations, and then calculated Kₘ/Vₘₐₓ and K_{cat}/Kₘ by Lineweaver-Burk equation.

Results and Discussion

Establishment of the GC-enrichment mutagenesis method Isotope-labeled dUTP has been widely used in random substitution of dTTP in DNA probe preparation. According to the rules of codon-anticodon recognition (Lewin, 2000), U can pair with A or G, respectively, during the process of mRNA translation. U:G pairs are also common in RNA duplex structures. We reckoned that such a U:G pairing potential could be exploited to increase the GC content of the target genes using error-prone PCR approach. Hence, two independent PCR reactions were conducted in the presence of dUTP, but lacking dTTP and dCTP, respectively. The result showed that the PCR product was well amplified in the absence of dTTP, but no PCR band was detected in the reaction lacking dCTP (data no shown), indicating that T can be replaced with U completely by Taq DNA polymerase. We then used the chimeric ‘DNA’ containing U instead of T as the template to conduct the second round PCR with normal concentrations of dNTP (200 μM each). No significant base changes were detected by sequencing the amplified fragments. The data suggest that Taq DNA polymerase favours U:A pairing under standard PCR conditions. To promote U:G pairing, we enhanced the polar environment of the reaction system by adding a highly hydrophilic molecule PEG3500 to the reaction buffer. In addition, we decreased dATP concentration to 8-20 μM, and increased dGTP concentration to 500 μM during the second round PCR (Materials and Methods, Table 2). Analysis of more than 33,920 bases amplified using this protocol showed that these modifications increased the base mutation rate up to 1.3%, and amino acid mutation rate up to 2.5% (Table 2). No nucleotide insertion or deletion has been detected. Among those mutated bases, more than 98% were AT to GC conversion (Table 2). The normal error rate of Taq DNA polymerase is about 0.01% (Tindall and Kunkel, 1988; Barnes, 1992). This GC-enrichment method thus represents over 130-fold increment in the rate of mutation, which is dominantly AT bias.

TABLE 2
Base substitution frequency of GC-enrichment mutagenesis*

| dATP | | | | Amino acid | Relative AT |
|---|---|---|---|---|---|
| concentration | | Observed base | Base mutation | mutation | substitution |
| (μM) | Analyzed bases | transition | frequency (%) | frequency (%) | frequency (%) |
| 8 | 5510 | | 1.31 | 2.53 | 98.6 |
| | A: 1716 | A to G: 47 | 2.74 | | |
| | T: 1228 | T to C: 24 | 1.95 | | |
| | G: 1130 | — | — | | |
| | C: 1436 | C to T: 1 | 0.07 | | |
| 12 | 9780 | | 1.17 | 2.15 | 99.1 |
| | A: 3046 | A to G: 58 | 1.90 | | |
| | | A to T: 1 | 0.03 | | |
| | | A to C: 1 | 0.03 | | |
| | T: 2180 | T to C: 51 | 2.34 | | |
| | | T to A: 2 | 0.09 | | |
| | G: 2004 | G to A: 1 | 0.05 | | |
| | C: 2550 | — | — | | |
| 16 | 9960 | | 0.95 | 1.97 | 97.8 |
| | A: 3102 | A to G: 51 | 1.64 | | |
| | T: 2220 | T to C: 38 | 1.71 | | |
| | | T to A: 3 | 0.14 | | |
| | G: 2041 | G to A: 1 | 0.05 | | |
| | C: 2597 | C to T: 1 | 0.04 | | |
| 20 | 8670 | | 0.84 | 1.88 | 98.6 |
| | A: 2700 | A to G: 35 | 1.30 | | |
| | | A to T: 2 | 0.07 | | |
| | T: 1933 | T to C: 34 | 1.76 | | |
| | | T to A: 1 | 0.05 | | |
| | G: 1776 | — | — | | |
| | C: 1226 | C to T: 1 | 0.04 | | |

*The data were obtained by analysis of the 64 amplified PCR fragments using the GFP gene as template.

Effect of Base Substitution on Amino Acid Composition of Protein

We analyzed the influence of enhanced GC content on amino acid composition. For the convenience of discussion, we defined the amino acid residues generated by the GC-enrichment mutagenesis as gain mutation, and the amino acid residues eliminated by the mutagenesis as lose mutation (Table 3). From the 13040 amino acids analyzed, we found that the most frequent gain mutations were RGPSAE (Arg, Gly, Pro, Ser, Ala, and Glu) by the GC-enrichment mutagenesis, and the most common lose mutations were KLFYN (Lys, Leu, Phe, Tyr, Asn) (Table 3). Therefore, changes in composition of DNA would result in changes in amino acid composition as well. Noticeably, among those gain mutation favourites, i.e., RGPSAE, Arg and Glu are charged residues while Pro, Ala, and Gly belong to hydrophobic residue group. Previous studies indicated that the properties most correlated with the proteins of the thermophilic bacteria are high percentages of Glu, Arg, and Lys, and low in uncharged polar residues in proteins (Haney et al., 1999; Vieille and Zeikus, 2001; Tekaia et al., 2002). The results in Table 3 showed that the gain mutation of Arg was largely due to lose mutation in Lys (charged), and H is (polar). Arg residue is commonly rich in hypertheromophilic proteins and believed to be better adapted to high temperatures than Lys residue because of its high pKa and its resonance stabilization (Vieille and Zeikus, 2001). Proline, which has the lowest conformational entrophy than other amino acid residues, was proved useful to improve thermostability of proteins by numerous experiments (Vieille and Zeikus, 2001; Zhang et al., 2002). Our data are consistent with the genome analysis studies that amino acid composition pattern is essentially driven by GC content in DNA (Singer & Hickey, 2000; Tredj et al., 2002). Moreover, the GC-enrichment mutagenesis appears to have the tendency to increase the lumped pool of the amino acid residues that are associated with protein thermostability.

Table 3. Change of amino acid composition caused by GC-enrichment mutagenesis

| | | Uncharged polar | | | | Nonpolar | | | | | | | | | | | | Charged | | | | |
|---|---|---|---|---|---|---|---|---|---|---|---|---|---|---|---|---|---|---|---|---|---|---|
| | | From | | | | | | | | | | | | | | | | | | | | |
| To | | S | Q | N | T | C | G | A | H | M | Y | F | V | L | P | I | W | K | R | D | E | Total (−) |
| Uncharged polar | S | | | | 2 | | 2 | | | | | | | | 5 | | | | | | | 9 |
| | Q | | | | | | | | | | | | | | | | | | 3 | | | 3 |
| | N | 15 | | | 1 | | | | 1 | | | | | | | 2 | | 1 | | | | 20 |
| | T | 1 | | | | | | | | | | | | | 1 | | | | | | | 2 |
| Nonpolar | C | | | | | | | | | | 1 | | | | | | | | 1 | | | 2 |
| | G | | | | | 1 | | | | | | | | | | 1 | | | 1 | | | 3 |
| | A | | | | 2 | | | | | | | | 1 | | | | | | | 2 | | 5 |
| | H | | | | | | | | | | 1 | | | 1 | | | | | 12 | | | 14 |
| | M | | | | 1 | | | | | | | | | | | | | | | | | 1 |
| | Y | | | | | 13 | | 8 | | | | | 1 | | | | | | | 2 | | 24 |
| | F | 9 | | | | | | | | | 1 | | | 14 | 1 | | | | | | | 24 |
| | V | | | | | | | 15 | | | | | | | | 1 | | | | | | 16 |
| | L | 3 | | | | | | | 5 | | | | | | 24 | | | | | | | 32 |
| | P | 1 | 1 | | | | 1 | | | | | | | 1 | | | | 1 | | | | 5 |
| | I | | | | 8 | | | | | | | | 8 | | | | | | | | | 16 |
| | W | | | | | | | | | | | | | | | | | | | | | |
| Chargeddd | K | | | | | | | | | | | | | | | | | | 28 | | 17 | 45 |
| | R | | | | | | 4 | | 1 | | | | | | | | | | | | | 5 |
| | D | | | 1 | | | 14 | | | | | | | | | | | | 1 | | | 16 |
| | E | | | | | | 11 | | | | | | | | | | | | | 2 | | 13 |

| | | | | | | | | | | | | | | | | | | |
|---|---|---|---|---|---|---|---|---|---|---|---|---|---|---|---|---|---|---|
| Total | 29 | 1 | 1 | 14 | 14 | 32 | 23 | 7 | 3 | 10 | 16 | 31 | 4 | 2 | 46 | 6 | 17 | 256 |
| (+) | | | | | | | | | | | | | | | | | | |

*The data were compiled from the analysis of 64 variant sequences (11,306 amino acid residues).
Directed Evolution of Albicidin Detoxifying Enzyme

To test whether this GC-enrichment mutagenesis approach can be used to improve enzyme activity, pQE60-albD containing albD was used as a template to generate an albD mutant library. From 63,000 clones, two albD variants, designated AlbD-M1 and AlbD-M2 with high enzymatic activity were identified based on their resistance to albicidin on plate assay. Sequence analysis of the two variants showed that there is a non-synonymous mutation at Ser40 (Ser to Gly) in AlbD-M1, and three non-synonymous mutations at Glu25 (Glu to Arg), Lys27 (Lys to Glu) and Ser40 (Ser to Gly) in AlbD-M2. For quantitative analysis and kinetic studies, AlbD and its variants were expressed as GST (glutathione S-transferase) fusion proteins, which were selectively bound to Glutathione Sepharose 4B affinity columns. The pure recombinant AlbD and variants were released from the columns by thrombin digestion (Materials and Methods). As the chemical structure of albicidin has not yet been identified, we compared the relative enzyme activity of AlbD and its two variants using the purified albicidin. Results showed that the detoxification activity of the evolved variants AlbD-M1 and AlbD-M2 was increased by 3- and 1.7-fold, respectively, in comparison with the wild type AlbD (Table 3).

Kinetic Characterization of Wild Type AlbD and Variants

The kinetic parameters of AlbD and variants were determined using 5 p-nitrophenyl compounds as substrates (Table 4). The results showed that AlbD has a wide substrate spectrum showing strongest catalytic activity against p-nitrophenyl butyrate (C4), followed by p-nitrophenyl valerate (C5), p-nitrophenyl caproate (C6), p-nitrophenyl propionate (C3), and p-nitrophenyl acetate (C₂) (Table 5). Mutations in the two AlbD variants in general did not change the substrate specificity, but there were significant increments in enzyme activity. AlbD-M1 exhibited 30-, 13-, and 43-fold increase in the K_{cat}/Kₘ, value on p-nitrophenyl acetate, p-nitrophenyl caproate, and p-nitrophenyl butyrate, respectively. The other variant AlbD-M2 showed a moderate 2-fold increase in K_{cat}/Kₘ value for p-nitrophenyl compounds with fatty acid chain of C₂, C₃, and C₄. The data indicate that substitution of Ser40 with Gly significantly increased the catalytic efficiency of the enzyme, whereas the substitution of Glu25 with Arg, and Lys27 with Glu simultaneously reduced the positive effect of Ser40Gly on catalytic efficiency. It could not rule out at this stage that Glu25 and Lys27 might also play a role in enzyme activity.

TABLE 4
The enzyme activity of AlbD and its variants

| | | Enzyme activity |
|---|---|---|
| Enzyme | Mutation | (units)* |
| AlbD | — | 87.4 |
| AlbD-M1 | Ser40Gly (A118G) | 263.4 |
| AlbD-M2 | Glu25Arg (A74G) | 147.1 |
| | Lys27Glu (A79G) | |
| | Ser40Gly (A118G) | |

*One unit of AlbD enzyme activity is defined as digested albicidin (ng) per min per μM enzyme. The data are means of triplicate repeats.

TABLE 5
Kinetic parameters of AlbD and its variants AlbD-M1 and AlbD-M2

| | | | | K_{cat}/Kₘ |
|---|---|---|---|---|
| Enzyme | Substrate | Kₘ (M) | K_{cat} (S⁻¹) | (M⁻¹ · S⁻¹) |
| AlbD | p-nitrophenyl caproate | 2.5 × 10⁻³ | 1.1 × 10⁻¹ | 55.6 |
| | p-nitrophenyl velerate | 1.7 × 10⁻³ | 2.8 × 10⁻¹ | 164.7 |
| | p-nitrophenyl butyrate | 1.9 × 10⁻⁴ | 1.0 × 10⁻¹ | 526 |
| | p-nitrophenol propionate | 2.2 × 10⁻¹ | 8.3 × 10⁻² | 3.8 × 10⁻¹ |
| | p-nitrophenol acetate | 6.8 × 10⁻² | 3.8 × 10⁻³ | 5.6 × 10⁻² |
| AlbD- | p-nitrophenol caproate | 2.5 × 10⁻⁴ | 4.2 × 10⁻¹ | 1680 |
| M1 | p-nitrophenol velerate | 4.0 × 10⁻⁴ | 8.8 × 10⁻¹ | 2200 |
| | p-nitrophenol butyrate | 1.1 × 10⁻⁵ | 2.5 × 10⁻¹ | 22727 |
| | p-nitrophenol propionate | 1.2 × 10⁻¹ | 2.7 × 10⁻¹ | 2.3 |
| | p-nitrophenol acetate | 9.4 × 10⁻² | 9.1 × 10⁻² | 9.6 × 10⁻¹ |
| AlbD- | p-nitrophenol caproate | 6.6 × 10⁻² | 1.3 × 10⁻¹ | 2.0 |
| M2 | p-nitrophenol butyrate | 2.3 × 10⁻⁵ | 2.8 × 10⁻² | 1217 |
| | p-nitrophenol propionate | 1.3 × 10⁻¹ | 1.0 × 10⁻¹ | 7.6 × 10⁻¹ |
| | p-nitrophenol acetate | 1.3 × 10⁻² | 1.5 × 10⁻² | 1.2 |

*Kₘ and Vₘₐₓ were determined by Lineweaver-Burk equation. The data are means of triplicate repeats.

The Structural and Functional Implications of the Ser40Gly Mutation

We are curious to know why Ser40Gly mutation could result in such a significant increase in catalytic efficiency. To probe the structure-function relationship, we compared AlbD with other esterases and lipases. AlbD shares less than 25% homology with other enzymes except several conserved short stretches of sequences. Besides the previously identified “¹⁰³G(A)xSxG¹⁰⁷” (AlbD numbering) motif, which is a conserved motif of catalytic importance in serine hydrolase family including lipases and esterases (Zhang et al., 1997), we found a “³⁴L(I,V)xxxGxxG⁴¹” (AlbD numbering) motif which is also highly conserved among various esterases and lipases (FIG. 2). L (Leucine), I (isoleucine), and V (valine) each contains a very similar hydrophobic side chain, therefore, they are functionally exchangeable. The general feature of the motif is highly hydrophobic; most of the amino acids within the motif contain hydrophobic side chains (FIG. 2).

We further examined the protein 3-D structure information to investigate the potential role of “³⁴L(I,V)xxxGxxG⁴¹”, motif. Protein structure analysis of the lipase of Bacillus stearothermophilus (Bse in FIG. 2) showed that the “³⁴L(I,V)xxxGxxG⁴¹” motif is located close to the catalytic triad (Tyndall et al., 2002). Superposition of a lipase of Pseudomonas cepacia (Pcl in FIG. 2) with several lipases from other sources showed that 5 amino acids within the motif are involved in formation of the oxyanion hole and in stabilizing the region around the oxyanion hole (Schrag et al., 1997). In serine hydrolases, catalytic triad together with the oxyanion hole form the active center. The catalytic role of the oxyanion hole is generally established to be in stablizing high-energy intermediates and the transition state through hydrogen bonding (Zhang, Y. et al., 2002). Significantly, both AlbD-M1 and AlbD-M2 contain a Ser40Gly mutation which is located within the “³⁴L(I,V)xxxGxxG⁴¹”, motif. Our findings are consistent with the protein structure analysis, and highlight that changing the amino acid within the motif could significantly influence the enzyme catalytic efficiency. This could be a promising clue not only for investigation of the structure-function relationship and but also for protein engineering of lipases and esterases, which are widely used in industry and in biotechnological applications (Jaeger et al., 1999; Bornscheuer, 2002).

### REFERENCES

Barnes, W. M. The fidelity of Taq polymerase catalyzing PCR is improved by an N-terminal deletion. Gene 112, 29-35 (1992).
Bernardi, G. Isochores and the evolutionary genomics of vertebrates. Gene 241, 3-17 (2000).
Bornscheuer, U. T. Microbial carboxyl esterases: classification, properties and application in biocatalysis. FEMS Microbiol. Rev. 26, 73-81.
Buchholz, F. & Stewart, F. Alteration Cre recombinase site specificity by substrate-linked protein evolution. Nat. Biotech. 19, 1049-1052 (2001).
Chemy, J. R., Lamsa, M. H., Schneider, P., Vind, J., Svendsen, A., Jones, A. & Pedersen, A. H. Directed evolution of a fungal peroxidase. Nat. Biotech. 17. 379-384 (1999).
Coco, W. M., Encell, L. P., Levinson, W. E., Loomis, A. K., Licato, L. L., Arensdorf, J. J., Sica, Nicole; Pienkos, Philip T; Monticello et al. Growth factor engineering by degenerate homoduplex gene family recombination, Nature Biotech. 20, 1246-1250 (2002).
Crameri, A., Raillard, Sun-Ai., Bermudez, E. & Stemmer, W. P. C. DNA shuffling of a family of genes from diverse species accelerates directed evolution. Nature. 391, 288-291 (1998).
Derewenda, Z. S & Sharp, A. M. News from the interface: the molecular structures of triacylglyceride lipases, Trends Biochem. Sci. 18, 20-25 (1993).
Flores, H. & Ellington, A. D. Increasing the thermal stability of an Oligomeric protein, beta-glucuronidase. J. Biol. Chem. 315, 325-337 (2002).
Fraser, C. M., Norris, S. J., Weinstock, G. M. et al. Complete genome sequence of Treponema pallidum, the syphilis spirochete. Science 281, 375-388 (1988).
Fromant, M., Blanquet, S. & Plateau, P. Direct random mutagenesis of gene-sized DNA fragments using polymerase chain reaction. Anal. BiocheM. 224, 347-353 (1995).
Galtier, N., Piganeau, G., Mouchiroud, D. & Duret, L. GC-content evolution in mammalian genomes: the biased gene conversion hypothesis. Genetics 159, 907-911 (2001).
Giver, L., Gershenson, A., Freskgard. Per-Ola. & Arnold, F. H. Directed evolution of a thermostable esterase. Proc. Natl. Acad. Sci. USA 95, 12809-12813 (1998).
Glaser, P., Frangeul, L., Buchrieser, C. et al. Comparative genomics of Listeria species. Science. 294, 849-852 (2001).
Glieder, A., Farinas, E. T. & Arnold, F. H. Laboratory evolution of a soluble, self-sufficient, highly active alkane hydroxylase. Nat. Biotech. 20, 1135-1139 (2002).
Haney, P. J., Badger, J. H., Buldak, G. L., Reich, C. I., Woese, C. R. & Olsen, G. L. Thermal adaptation analysed by comparison of protein sequences form mesophilic and extremely thermophilic Methanococcus species. Proc. Natl. Acad. Sci. USA 96, 3578-3583 (1999).
Herbert, A. & Rich, A. Left-handed Z-DNA: structure and function. Genetica 106, 37-47 (1999).
Jaeger, K. E., Dijkstra, B. W. & Reetz, M. T. Bacterial biocatalysts: molecular biology, three-dimensional structures, and biotechnological applications of lipases. Annu. Rev. Microbiol. 53, 315-351 (1999).
Kugimiya, W; Otani, Y; Hashimoto, Y. Molecular cloning and structure of the gene for esterase from a thermophilic bacterium, Bacillus stearothermophilus IFO 12550, Biosci. Biotech. Biochem. 56, 2074-2075 (1992).
Kunst, F., Ogasawara, N., Moszer, I. et al. The complete genome sequence of the Gram-positive bacterium Bacillus subtilis. Nature 390, 249-256 (1997).
Kuroda, M., Ohta, T., Uchiyama, I. et al. Whole genome sequencing of meticillin-resistant staphylococcus aureus. Lancet. 357, 1218-1219 (2001).
Leong, S. R., Chang, J. C. C., Ong, R., Dawes, G. D., Stemmer, W. P. C. & Punnonen, J. Optimized expression and specific activity of IL-12 by directed molecular evolution. Proc. Natl. Acad. Sci. USA 100, 1163-1168 (2003).
Lewin, B. Chapter 7, Using the Genetic Code. In Genes VII, Oxford University Press Inc., New York, pp 167-190 (2000).
Leung, D. W., Chen, E. & Goeddel, D. V. A method for random mutagenesis of a defined DNA segment using a modified polymerase chain reaction. Technique 1, 11-15 (1989).
Miyazaki, K., Wintrode, P. L., Grayling, R. L., Rubingh, D. N. & Arnold, F. H. Directed evolution study of temperature adaptation in a psychrophilic enzyme. J. Mol. Biol. 297, 1015-1026 (2000).
Narum, D. L., Kumar, S., Rogers, W. O., Fuhrmann, S. R., Liang, H., Oakley, M., Taye, A., Sim, B. K. L & Hoffman, S. L. Codon optimization of gene fragments encoding Plasmodium falciparum merzoite proteins enhances DNA vaccine protein expression and immunogenicity in mice. Infect. Immun. 69, 7250-7253 (2001).
Nelson, K. E., Clayton, R. A., Gill, S. R. Evidence for lateral gene transfer between Archaea and Bacteria from genome sequence of Thermotoga maritima. Nature 399, 323-329 (1999).
Ness, J. E., Kim, S., Gottman, A., Pak, R., Krebber, A., Borchert, T. V., Govindarajan, S., Mundorff, E. C. & Minshull, J. Synthetic shuffling expands functional protein diversity by allowing amino acids to recombine independently. Nat. Biotech. 20, 1251-1255 (2002).
Ostermeier, M., Shim, J. H. & Benkovic, S. J. J. A combinatorial approach to hybrid enzyme independent of DNA homology. Nat. Biotech. 17, 1205-1209 (1999).
Ou, H-Y., Guo, F-B. & Zhang, C-T. Analysis of nucleotide distribution in the genome of Streptomyces coelicolor A3(2) using the Z curve method. FEBS Lett. 540, 188-194 (2003).
Perlak, F. J., Fuchs, R. L., Dean, D. A., McPherson, D. S. & Fischhoff, D. A. Modification of the coding sequence enhances plant expression of insect control protein genes. Proc. Natl. Acad. Sci. USA 88, 3324-3328 (1991).
Rothman, S. C. & Kirsch. How does an enzyme evolution in vitro compare to naturally occurring homologs possessing the targeted function? Tyrosine aminotransferase from aspartate aminotransferase. J. Mol. Biol. 327, 593-608 (2003).
Schrag, J. D., Li, Y., Cygler, M., Lang, D., Burgdorf, T., Hecht, H-J., Schmid, R., Schomburg, D., Rydel, T. J., Oliver, J. D. et al. The open conformation of a Pseudomonas lipase. Structure 5, 187-202 (1997).
Shimshek, D. R., Kim, J., Hübner, M. R., Spergel, D. J., Buchholz, F., Casanova, E., Stewart, A. F., Seeburg, P. H. & Sprengel, R. Codon-improved cre recombinase (iCre) expression in the mouse. Genesis 32, 19-26 (2002).
Singer, G. A. C. & Hickey, D. A. Nucleotide bias causes a genome wide bias in the amino acid composition of protein. Mol. Biol. Evol. 17, 1581-1588 (2000).
Spee, J. H., de Vos, W. M. & Kuipers, O. P. Efficient random mutagenesis method with adjustable mutation frequency by use of PCR and dITP. Nucleic. Acids Res. 21, 777-778 (1993).
Stemmer, W. P. C. Rapid evolution of a protein in vitro by DNA shuffling. Nature 370, 389-391 (1994).
Takami, H., Nakasone, K., Takaki, Y. et al. Complete genome sequence of the alkaliphilic bacterium Bacilli halodurans and genomic sequence comparison with Bacillus subtilis. Nucleic Acids Res. 28, 4317-4331 (2000).
Tekaia, F., Yeramian, E. and Dujon, B. Amino acid composition of genomes, lifestyles of organisms, and evolutionary trends: a global picture with correspondence analysis. Gene 297, 51-60 (2002).
Tindall, K. R. & Kunkel, T. A. Fidelity of DNA synthesis by the thermus aquaticus DNA polymerase. Biochem. 27, 6008-6013 (1988).
Tredj, T., Yeramian, E. & Dujon, B. Amino acid composition of genomes, lifestyles of organism, and evolutionary trends: a global picture with correspondence analysis. Gene. 297, 51-60 (2002).
Tyndall, J. D. A., Sinchaikul, S., Fothergill-Gilmore, L. A., Taylor, P & Walkinshaw, M. D. Crystal structure of a thermostable lipase from Bacillus stearothermophilus P1. J. Mol. Biol. 323, 859-869 (2002).
Valendic, M. L. & McDonald, J. A. Codon optimization markedly improves doxycycline regulated gene expression in the mouse heart. Transgenic Res. 10, 269-275 (2001).
Vieille, C. & Zeikus, G. J. Hyperthermophilic enzyme: sources, uses, and molecular mechanisms for thermostability. Microbiol. Mol. Biol. Rev. 65.1-43 (2001).
Vinogradov, A. E. DNA helix: the importance of being GC-rich. Nucleic. Acids. Res. 31, 1838-1844 (2003).
Wintrode, P. L., Zhang, D., Vaidehi, N., Arnold, F. H. & Goddard III, W. A. Protein dynamics in a family of laboratory evolved thermophilic enzymes. J. Mol. Biol. 327, 745-757 (2003).
Xia, G., Chen, L., Sera, T., Fa, M., Schulz, P. G. & Romesberg, F. E. Directed evolution of novel polymerase activities: Mutation of a DNA polymerase into an efficient RNA polymerase. Proc. Natl. Acad. Sci. USA 99, 6597-6602 (2002).
Yadava, A. & Ockenhouse, C. F. Effect of codon optimization on expression levels of a functionally folded malaria vaccine candidate in prokaryotic and eukaryotic expression systems. Infect. Immun. 71, 4961-4969 (2003).
Yang, J. K., Park, M. S., Waldo, G. W. & Suh, S. W. Directed evolution approach to a structural genomics project: Rv2002 from Mycobacterium tuberculosis. Proc. Natl. Acad. Sci. USA 100, 455-460 (2003).
Yano, T., Oue, S. & Kagamiyama. Directed evolution of an aspartate aminotransferase with new substrate specificities. Proc. Natl. Acad. Sci. USA 95, 5511-5515 (1998).
Yokobayashi, Y., Weiss, R. & Arnold, F. H. Directed evolution of a genetic circuit. Proc. Natl. Acad. Sc. USA 99, 16587-16591 (2002).
Zaccolo, M. & Gherardi, E. The effect of high-frequency random mutagenesis on in vitro protein evolution: a study on TEM-1. J. Mol. Biol. 285, 775-783 (1999).
Zaccolo, M., Williams, D. M., Brown, D. M. & Gherardi. An approach to random mutagenesis of DNA using mixture of triphosphate derivatives of nucleoside analogues. J. Mol. Biol. 255, 589-603 (1996).
Zhang, D. H., Li, X. and Zhang, L. H. Isomaltulose synthase from Klebsiella sp. strain LX3: gene cloning and characterization and engineering of thermostability. Appl. Environ. Microbiol. 68, 2676-2682 (2002).
Zhang, J-H., Dawes, G. & Stemmer, W. P. C. Directed evolution of a fucosidase from a galactosidase by DNA shuffling and screening. Proc. Natl. Acad. Sci. USA 94, 4504-4509 (1997).
Zhang, L. H., Xu, J. L. & Birch, R. G. Engineered detoxification confers resistance against a pathogenic bacterium. Nat. Biotech. 17, 1021-1024 is (1999).
Zhang, L. H. & Birch, R. G. The gene for albicidin detoxification from pantoea dispersa encodes an esterase and attenuates pathogenicity of Xanthomonas albilineans to sugarcane. Proc. Natl. Acad. Sci. USA 94, 9984-9987 (1997).
Zhang, L. H., Xu, J. & Birch, R. G. Factors affecting biosythesis by Xanthomonas albilineans of albicidin antibiotics and phytotoxins. J. Appl. Microbiol. 85, 1023-1028 (1998).
Zhang, Y. Kua, J. and McCammon, J. A. Role of the catalytic triad and oxyanion hole in acetylcholinesterase catalysis: an ab initio QM/MM study. J. Am. Chem. Soc. 124, 10572-10577 (2002).
Zhao, H., Giver, L., Shao, Z., Affholter, J. A. & Arnold, F. H. Molecular evolution by staggered extension process (STEP) in vitro recombination. Nat. Biotech. 16, 258-261 (1998).

1. A method for enriching the GC base pair content of a DNA molecule the method comprising the steps of (a) providing a DNA template molecule in which at lest some of the A residues are base paired with U residues and (b) replicating the DNA template molecule provided in step (a) under conditions in the replication reaction medium in which at least some of the U residues base pair with a G residue.

2. A method according to claim 1 wherein the DNA template molecule in (a) is produced by replicating a first template DNA molecule in the presence of dUTP so that at lest some of the T residues of the first template are replaced by U residues to form a second template molecule.

3. A method according to claim 1 comprising the steps of (1) providing a first template DNA molecule, (2) replicating the first template DNA molecule in the presence of dUTP so that at least some of the T residues of the first template are replaced by U residues to form a second template molecule and (3) replicating the DNA template molecule produced in step (2) under conditions in the replication reaction medium in which at least some of the U residues base pair with a G residue.

4. A method according to claim 1 wherein the conditions in which at least some of the U residues base pair with a G residue are provided by an agent which is able to increase the polarity of the replication reaction medium and/or act as a local dehydrating agent.

5. A method according to claim 4 wherein an excess of dGTP over dATP or dCTP or dTTP is present in the reaction medium during replication.

6. A method according to claim 4 wherein the agent which is able to increase the polarity of the reaction medium and/or act as a local dehydrating agent is polyethylene glycol (PEG).

7. A method according to claim 6 wherein the PEG is PEG300-PEG8000.

8. A method according to claim 1 wherein the DNA replication is by a polymerase chain reaction.

9. A method according to claim 1 wherein the DNA molecule whose GC base pair content is to be enriched comprises all or part of a natural gene or cDNA.

10. A method according to claim 9 wherein the gene or cDNA has a GC base pair content lower than 50%.

11. A method according to claim 9 wherein the gene or cDNA encodes a polypeptide of interest.

12. A method according to claim 10 wherein the polypeptide is any of an enzyme, an antibody chain or an antigen.

13. A method according to claim 1 comprising the further step of cloning the DNA molecule whose GC base pair content has been enriched.

14. A method according to claim 13 wherein the DNA molecule whose GC base pair content has been enriched is cloned into an expression vector.

15. A method according to claim 1 comprising the further step of sequencing the DNA molecule whose GC base pair content has been enriched.

16. A method according to claim 15 comprising the further step of selecting those of the DNA molecules whose GC base pair content is has been enriched wherein the coding sense has been retained.

17. A method according to claim 15 comprising the further step of selecting those of the DNA molecules whose GC base pair content has been enriched wherein coding sense has been altered.

18. A method according to claim 11 comprising the further step of expressing a polypeptide from the gene or cDNA whose GC base pair content has been enriched.

19. A DNA molecule enriched for GC base pair content prepared by the method of claim 1.

20. A method for making a mutant polypeptide with altered properties compared to the polypeptide encoded by a given DNA molecule, the method comprising (a) enriching the GC base pair content of the DNA molecule according to the method of claim 1, (b) expressing the polypeptide encoded by the DNA molecule whose GC base pair content has been enriched in step (a), and (c) selecting a polypeptide with altered properties.

21. A method according to claim 20 wherein the polypeptide is any of an enzyme, an antibody chain or an antigen.

22. A method according to claim 21 wherein the polypeptide is an enzyme which has been selected in step (c) for improved catalytic properties.

23. A method according to claim 22 wherein the enzyme is encoded by the albD gene of Pantoea dispera.

24. A mutant polypeptide prepared by the method of claim 20.

25. A mutant AlbD polypeptide prepared by the method of claim 20 wherein Ser40 has been replaced by another amino acid residue.

26. A mutant AlbD polypeptide according to claim 25 wherein Ser40 has been replaced with Gly.

27. A mutant AlbD polypeptide according to claim 25 wherein Glu25 has been replaced by Arg, Lys27 has been replaced by Glu and Ser40 has been replaced by Gly.

28. A polynucleotide encoding the mutant AlbD polypeptide according to claim 25.

29. An expression vector containing a polynucleotide according to claim 28.

30. A transgenic plant containing a polynucleotide according to claim 28.

31. A kit of parts for enriching the GC base pair content of a DNA molecule in a replication reaction medium comprising (a) dUTP and (b) an agent which is able to increase the polarity of the replication reaction medium and/or act as a local dehydrating agent.

32. A kit of parts according to claim 31 wherein the agent which is able to increase the polarity of the replication reaction medium and/or act as a local dehydrating agent is a polyethylene glycol.

33. A kit of parts according to claim 31 further comprising other reagents for carrying out a DNA amplification reaction.

34. (canceled)

35. A method according to claim 5 wherein the agent which is able to increase the polarity of the reaction medium and/or act as a local dehydrating agent is polyethylene glycol (PEG).

36. A method according to claim 10 wherein the gene or cDNA encodes a polypeptide of interest.

37. A DNA molecule enriched for GC base pair content prepared by the method of claim 2.

38. A DNA molecule enriched for GC base pair content prepared by the method of claim 3.

39. A DNA molecule enriched for GC base pair content prepared by the method of claim 4.

40. A DNA molecule enriched for GC base pair content prepared by the method of claim 5.

41. A DNA molecule enriched for GC base pair content prepared by the method of claim 6.

42. A DNA molecule enriched for GC base pair content prepared by the method of claim 7.

43. A DNA molecule enriched for GC base pair content prepared by the method of claim 8.

44. A DNA molecule enriched for GC base pair content prepared by the method of claim 9.

45. A DNA molecule enriched for GC base pair content prepared by the method of claim 10.

46. A DNA molecule enriched for GC base pair content prepared by the method of claim 11.

47. A DNA molecule enriched for GC base pair content prepared by the method of claim 12.

48. A DNA molecule enriched for GC base pair content prepared by the method of claim 13.

49. A DNA molecule enriched for GC base pair content prepared by the method of claim 14.

50. A DNA molecule enriched for GC base pair content prepared by the method of claim 35.

51. A DNA molecule enriched for GC base pair content prepared by the method of claim 36.

52. A mutant polypeptide prepared by the method of claim 21.

53. A mutant polypeptide prepared by the method of claim 22.

54. A mutant polypeptide prepared by the method of claim 23.

55. A mutant AlbD polypeptide according to claim 26 wherein Glu25 has been replaced by Arg, Lys27 has been replaced by Glu and Ser40 has been replaced by Gly.

56. A kit of parts according to claim 32 further comprising other reagents for carrying out a DNA amplification reaction.

57. A mutant AlbD polypeptide prepared by the method of claim 21 wherein Ser40 has been replaced by another amino acid residue.

58. A mutant AlbD polypeptide prepared by the method of claim 22 wherein Ser40 has been replaced by another amino acid residue.

59. A mutant AlbD polypeptide prepared by the method of claim 23 wherein Ser40 has been replaced by another amino acid residue.
